(19) United States
(12) Patent Application Publication
(10) Pub. No.: US 2001/0056265 A1
(43) Pub. Date: Dec. 27, 2001

(54) Syringe for medical use

(76) Inventors: Jochen Heinz, Vendersheim (DE); Michael Spallek, Inqelheim (DE); Arthur Fabian, Mainz (DE)

Correspondence Address: Walter Ottesen, Patent Attorney, P. O. Box 4026, Gaithersburg, MD, 20885-4026, US

(21) Appl. No.: 09/808,027

(22) Filed: Mar. 15, 2001

(30) Foreign Application Priority Data
Mar. 15, 2000 (DE) 100 12 574.3

(51) Int. Cl. A61M 5/315

(52) U.S. Cl. 604/227

(57) ABSTRACT

Syringes for medical purposes have syringe bodies which typically have a syringe body (2) on which a syringe head (3) is formed at the outlet end. A finger support (1) is at the other end. This finger support has an enlarged support surface in two mirror-symmetrical opposite-lying regions. To increase the support surface further, the finger support (1) is so configured that the two regions (1a, 1b) have a parabola-shaped configuration whose respective apexes are directed away from the syringe body (2). The two regions go over one into the other in the region (1c, 1d) of the mirror axis in an arcuate shape. In this way, the ergonomic manipulation of the syringes is improved and a high packing density of the syringes when magazining is made possible.

### FIELD OF THE INVENTION

The invention relates to a syringe for medical purposes. The syringe has a syringe head on which a closeable syringe head is formed at the output end. The syringe body has a finger support at the other end thereof and this finger support has an enlarged support surface in two mirror-symmetrical opposite-lying regions.

### BACKGROUND OF THE INVENTION

Syringes for medical purposes, in the sense of the invention, are syringes with which a pharmaceutical substance or a diagnostically effective substance can be dispensed by injection parenterally. The prefillable one-way syringes or so-called prefilled syringes become ever more significant. Such a disposable syringe avoids the otherwise usual transfer of the medical substance from ampules and injection vessels into the syringe. This transfer is not only time consuming but also makes possible a great number of sources of contamination.

Prefillable one-way syringes of tubular glass are the subject matter of DIN ISO 11040 Part 4. The dimensions of the syringes are fixed in this standard as are their configurations which are shown in longitudinal section in FIG. 5 herein. The head configuration of the syringe cylinder must be agreed to between manufacturer and customer. The head can exhibit a Luer cone which can be closed by a so-called tip-cap and, after removing the tip-cap, a needle unit can be pushed on. The head can also have an integrated needle which can be covered by a conventional needle cap.

A finger support is formed on the syringe cylinder and the configuration of this finger support is fixed by the above-mentioned standard and can be seen in FIG. 6. The finger support has a circularly-shaped base configuration and a segment is cut off at two of the opposite-lying regions so that in the two other circular arc regions, which lie opposite each other displaced by 90°, a relatively narrow support is given which is not ergonomically optimal. This support is so narrow that it is often necessary during use to increase the finger support surface by corresponding plastic parts which are clipped onto the syringe cylinder as disclosed, for example, in FIGS. 18 to 20 of European patent publication 0,738,517.

The configuration of the finger supports is similar in one-way syringes made of plastic in accordance with standard DIN 13 098 Part 1 as well as for prefillable syringes of plastic as they are known, for example, from German patent publication 4,438,360.

In addition to the disadvantage of the unfavorable ergonomic configuration of the finger support, an additional disadvantage is presented by the configuration of the finger support.

The syringes for medical purposes are typically first manufactured in a facility processing glass or plastic and, thereafter, are transported to a pharmaceutical facility where they are filled. A series of processing steps are necessary in the manufacture of the syringes as well as with the filling thereof. These processing steps include washing, sterilization, packaging, transporting, siliconizing, fabrication, et cetera. Thus, the processing of the disposable syringes (there known as syringe ampules) is known from the article of E. Venten et al entitled “Pharmazeutische Industrie” 40 No. 6, 1978, pages 665 to 671 as well as from the publication entitled “Die neue Verpackung”, Volume 31, 7/1978, pages 1062 to 1064.

Special problems are presented here in the manipulation, the transport and the storage of such filled as well as unfilled syringes. An essential reason is that these syringes are a mass produced article and, therefore they cannot be handled individually, but, for reasons of economics, they can only be handled by machines in orderly arrays or in so-called trays.

Corresponding arrangements are known in numerous variations and are disclosed, for example, in published international patent application WO 94/14484 and in European patent publication 0,790,063 showing the HYPAK® SCF® system. These arrangements typically have plastic aperture plates in which the syringes are loosely held with the syringes being suspended at their finger supports.

In order to provide the most efficient processing of the syringes possible, a high packing density of the syringes must, however, be achieved which, with the known systems, is achieved only inadequately because of the configuration of the finger supports.

### SUMMARY OF THE INVENTION

It is an object of the invention to configure the initially-described syringe so that it has an ergonomically favorable finger support and, at the same time, makes possible a very high packing density when magazining in the above-mentioned aperture plate (so-called nests).

The syringe of the invention is for medical use and includes: a syringe body having a first end defining a discharge end; a closeable syringe head formed on the discharge end; the syringe body having a second end lying opposite the first end; a finger support on the second end; the finger support defining an enlarged support surface subdivided into two mirror-symmetrical opposite-lying support-surface regions; each of the support surface regions having a parabolically-shaped configuration defining a parabolically-shaped edge and defining an apex directed away from the syringe body; the support surface regions conjointly defining a mirror axis and a transition region at the mirror axis; and, the transition region having oppositely-lying peripheral arcuate edges connecting the parabolically-shaped edges of the support-surface regions.

The finger support form of the invention permits a dense packaging of empty or filled syringes in transport devices in the form of the above-described aperture plates. Compared to the corresponding syringes in accordance with DIN ISO 11040-4, a significantly increased packaging density of the syringes of the invention results.

In addition, the finger supports are ergonomically significantly more favorable because they have a relatively large support surface which ensures a high reliability when manually grasping the same.

According to a feature of the invention, the finger support is formed as one piece on the syringe body. In this way, a simple manufacture of the syringe is possible.

In principle, however, the finger support can be a separate part which is joined to the syringe body with methods known per se such as with adhesive.

The syringe of the invention can be made of glass or, alternatively, also of plastic. As a plastic, cycloolefin copolymer (COC) or cycloolefin polymer (COP) are especially suitable and afford special advantages.

The syringe of the invention can be configured as a one-way syringe. The syringe of the invention can also be configured as a prefillable one-way syringe (disposable syringe) with a significant advantage.

Typically, the syringe body is configured to be cylindrical. However, syringes having cross-sectional configurations departing from the circular form are also conceivable.

In accordance with another feature of the invention, the syringe is so configured that the maximum outer radial dimensions of the syringe head (if required, including a corresponding closure) are not larger than the maximum radial dimension of the syringe body.

In this way, it is possible to magazine handle the syringes with the head suspended downwardly by means of the finger support in a transport device. This is possible even when the syringe head is already closed and the syringe is filled via the other open end and is then closed by means of a piston plug.

### BRIEF DESCRIPTION OF THE DRAWINGS

The invention will now be described with reference to the drawings wherein:

FIG. 1 is a plan view of a finger support of a one-way syringe configured in accordance with the invention;

FIG. 2 is a schematic perspective view of the basic configuration of a carrier plate for the collective transport of the syringes of the invention;

FIGS. 3A to 3D show respective cross sections of four different configurations of the clamping seat for holding the syringes in the bores of the carrier plate;

FIG. 4 is a plan view of a carrier plate charged with syringes having the finger support of the invention;

FIG. 5 is a side elevation view, partially in section, showing a standardized prefillable one-way syringe of glass;

FIG. 6 is a plan view of a standardized finger support of the standardized syringe of FIG. 5; and,

FIG. 7 is a schematic plan view of the carrier plate charged with syringes having standardized finger supports.

### DESCRIPTION OF THE PREFERRED EMBODIMENTS OF THE INVENTION

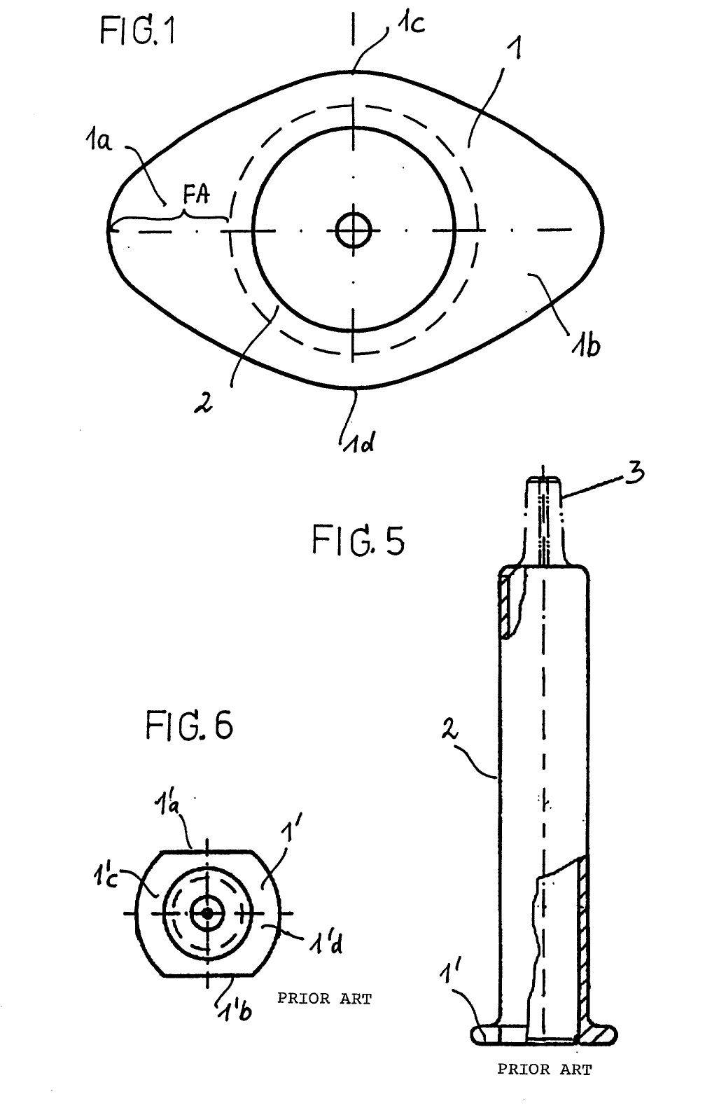

FIG. 1 shows the configuration of the finger support 1 of the invention for a prefillable one-way syringe in a greatly enlarged plan view. The prefillable syringe differs significantly from the configuration of the finger support 1′ (FIG. 6) of the known standardized disposable syringe which is shown in FIG. 5. The known so-called classic finger support of FIG. 6 has a circularly-shaped base configuration. Respective segments are cut off at opposite-lying regions 1′a and 1′b so that in the two other circular-arc regions 1′c and 1′d, which are offset 90° to the other two segments, only a relatively narrow support is provided which is not ergonomically optimal. This support also permits no optimal packaging density in the so-called formed recesses.

The known syringe of FIG. 5 includes a syringe cylinder 2 on which a syringe head 3 is formed, in the same manner as the finger support 1′ is formed typically on the syringe cylinder 2, that is, the finger support is connected as one piece therewith. The syringe head 3 can, as shown, be formed by a Luer cone which can be closed germ and liquid tight by a so-called tip-cap. The syringe head 3 can also have an integrated needle which is covered by a protective cap.

The special configuration of the finger support of the invention of FIG. 1 is configured such that it has two regions 1a and 1b having parabola-shaped contours. The two regions are symmetrical with respect to the center and are on two opposite-lying sides. In the center, the two regions extend into one another continuously in an arcuate shape (positions 1c and 1d). In this way, a relatively large distance FA is made available as a support for the fingers when utilizing the syringe. Furthermore, a very high packing density can be achieved which will be explained hereinafter.

Insofar as glass disposable syringes are concerned, the configuration of the syringe of the invention corresponds to that of FIG. 5 with a finger support of FIG. 1 formed preferably as one piece therewith.

The syringe therefore likewise comprises a cylindrical syringe body 2 and, at the head end, has a closure whose maximum diameter is not greater than the maximum diameter of the syringe body so that the syringe can be accommodated in the aperture holders with the head directed downwardly and the syringe supported at the finger support. The syringe body need not perforce be cylindrical and can also, as shown in published international patent application WO 99/22788, be multicornered in cross section.

The same as above applies when the syringe of the invention is manufactured of plastic.

As mentioned initially herein, the syringes of the above-mentioned type are typically magazine handled in aperture plates wherein the syringes are suspended at the finger supports and the syringe heads are directed downwardly.

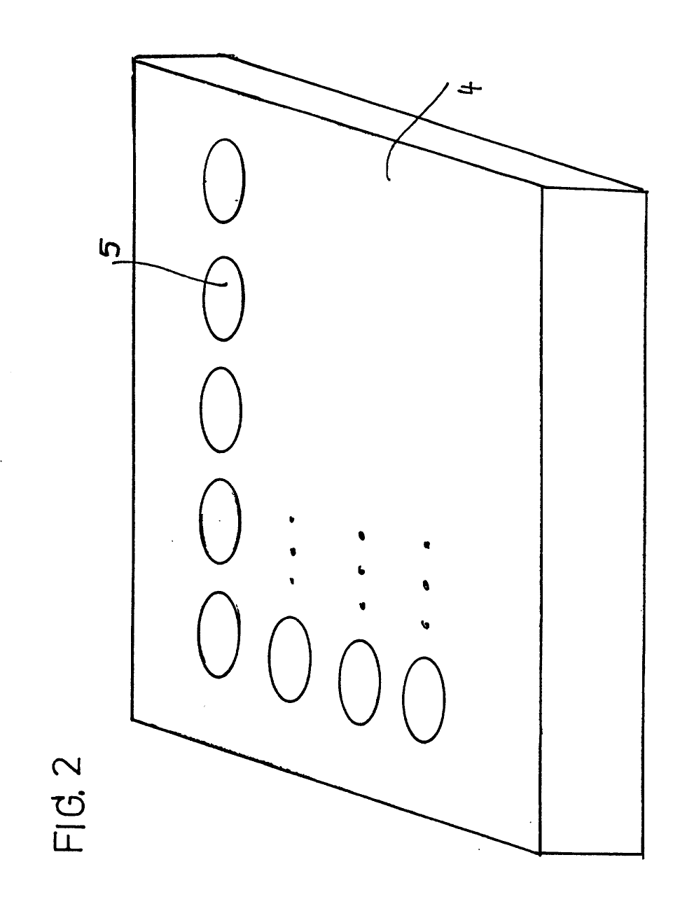

A transport device of this kind is shown in FIG. 2 which has a one-piece rectangularly-shaped carrier plate 4 made of foamed plastic which is resistant to bending. The plastic is preferably foamed polypropylene (PP) or polyester (PET) or like material as described, for example, in the exposition report for Tokyo Pack 1992 in “Neue Verpackung”, 12/92, pages 32 to 38.

In the carrier plate 4, circular bores 5 or even bores having multicornered cross sections are formed to accommodate the syringes 2. These bores 5 are circular and are in several rows of which only one complete row is shown. The bores 5 are formed already when the carrier plate is manufactured.

Preferably, the carrier plate 4 is so configured that it has a pore-free surface. This can typically be achieved via a subsequent surface treatment of the foamed carrier plate.

The diameter of the bores 5 are so matched to the diameters of the syringe cylinders 2 that the vessels are accommodated so as to be clamped and centered in the bore. In this way, the transport plate of the invention reliably prevents relative movements between the syringe bodies and the carrier plate 4 and is thereby suited also for scratch-sensitive syringes, for example, syringes made of brittle plastics.

In addition, the reliable guidance or holding of the syringes affords the advantage that no static charge and particle buildup can occur because of relative movements between the syringes and the carrier plate (nest).

Furthermore, the reliable guidance or holding prevents that the shaking movements, which are unavoidable during transport, lead to the situation that syringes, which jump upwardly, penetrate or damage the seal foil which serves to close off the entire unit and to prevent the danger of contamination. In the state of the art, protective intermediate foils or intermediate foams are utilized in order to prevent this contamination and this additional measure is unnecessary with the carrier plate according to the invention.

The transport plate can, if it can ensure this fixing, also be used for direct filling so that the syringes no longer have to be lifted out of the nest and centered and aligned in a complex manner for filling. This is in contrast to present-day practice. Also in contrast to the present-day state of the art, with the nest of the invention, a considerably higher filling capacity is achieved.

The transport plate according to the invention (when it has a closed-pore surface) can be autoclaved advantageously together with empty or filled syringes so that no reloading of the holders is necessary for this sterilization step.

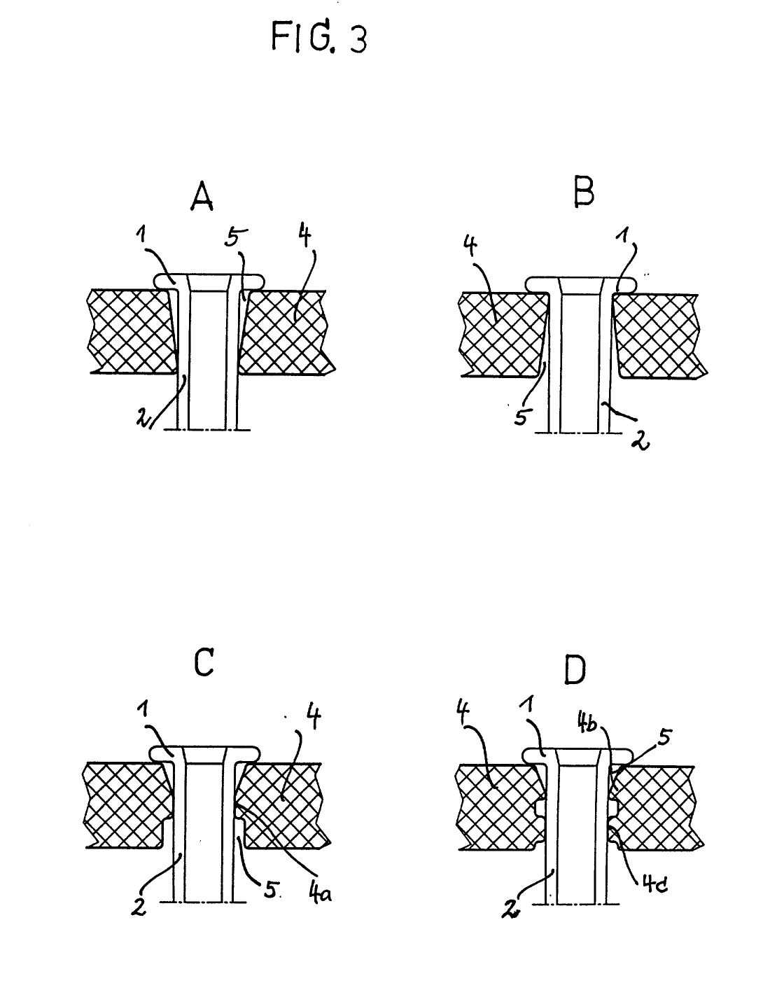

A cross section of the carrier plate 4 in the region of a bore 5 is shown in each of FIGS. 3A to 3D.

As shown in FIGS. 3A and 3B, the bores 5 are configured to be preferably conical for syringe bodies 2 having finger supports 1 which extend over the carrier plate 4 in the bore 5 so that the inserted syringe bodies are reliably fixed and centered. In this way, tolerances in the geometry of the syringe bodies are compensated.

The conical surface can be tapered downwardly as shown in FIG. 3A or tapered upwardly as shown in FIG. 3B.

The syringe body 2 can be clampingly held in the opening 5 of the carrier plate 4 in the manner shown in FIG. 3C so that a peripherally extending annular projection 4a is formed on the carrier plate 4 in the opening 5 having a diameter at its narrowest location which is somewhat less than the diameter of the syringe cylinder 2.

In the embodiment of FIG. 3D, two axially spaced peripherally extending annular shaped projections 4b and 4c are formed which further improves the reliable hold.

In a special embodiment of the transport plate according to the invention, the bores 5 are so configured that the guidance and clamping of the syringe body 2 takes place at three edges (point-shaped projections) which improves the self-centering effect and prevents a thermal isolation of a specific section of the syringe bodies. These edges are preferably at different axial elevations.

As an alternative to the configurations of FIGS. 3A to 3D, the bores 5 can also be so configured that the finger supports 1 lie recessed in the transport plate.

In a special embodiment, the transport plate 4 is made of foamed plastic which has been foamed in inert gases (for example, nitrogen, carbon dioxide, argon). This reliably prevents the formation of ozone gas during sterilization by energy-rich radiation (y radiation or electron radiation).

In accordance with one embodiment, the transport plate comprises foamed plastic which includes stiffening elements.

In an alternate embodiment, the transport plate includes a ribbed geometry for stiffening.

The carrier plate 4 can be used during manufacture and filling of the syringes for the most different handling operations. The carrier plate is also useable as an insert in a pan-shaped vessel as transport packaging for empty or filled syringes.

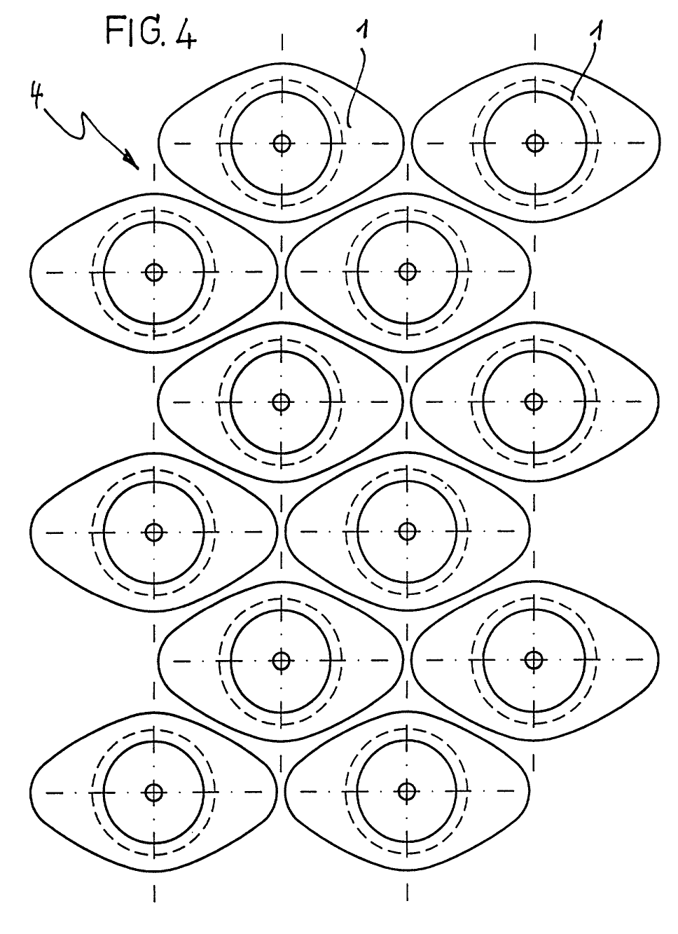

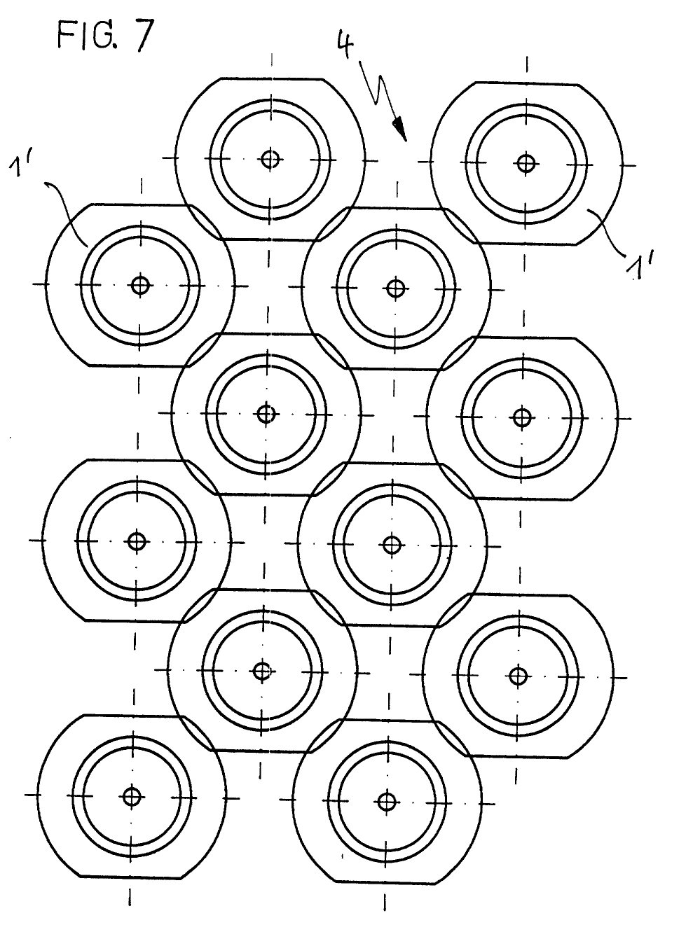

FIG. 4 shows a configuration of the carrier plate 4 having several rows of holes arranged offset with respect to each other so that a very high packing density can be achieved with syringes having the center-symmetric parabola-shaped finger supports configured in accordance with the invention. This makes manufacture and filling of the syringes more economical and thereby reduces cost. This high packaging density compared to the standard syringes is apparent visually when comparing FIG. 4 to FIG. 7 which leaves considerable gaps between the standardized finger supports 1′.

It is understood that the foregoing description is that of the preferred embodiments of the invention and that various changes and modifications may be made thereto without departing from the spirit and scope of the invention as defined in the appended claims.

What is claimed is:

1. A syringe for medical use, the syringe comprising:
a syringe body having a first end defining a discharge end;
a closeable syringe head formed on said discharge end;
said syringe body having a second end lying opposite said first end;
a finger support on said second end;
said finger support defining an enlarged support surface subdivided into two mirror-symmetrical opposite-lying support-surface regions;
each of said support surface regions having a parabolically-shaped configuration defining a parabolically-shaped edge and defining an apex directed away from said syringe body;
said support surface regions conjointly defining a mirror axis and a transition region at said mirror axis; and,
said transition region having oppositely-lying peripheral arcuate edges connecting the parabolically-shaped edges of said support-surface regions.

2. The syringe of claim 1, wherein said finger support is formed as one piece with said syringe body.

3. The syringe of claim 1, said syringe is made of glass.

4. The syringe of claim 1, wherein said syringe is made of plastic.

5. The syringe of claim 4, wherein said plastic is a cycloolefin copolymer (COC)or a cycloolefin polymer (COP).

6. The syringe of claim 1, wherein said syringe is configured as a one-way syringe.

7. The syringe of claim 1, wherein said syringe is configured as a prefillable one-way syringe.

8. The syringe of claim 1, wherein said syringe body is configured to be cylindrical.

9. The syringe of claim 1, wherein said syringe body has a maximum radial dimension and said syringe head likewise has a maximum radial diameter; and, said maximum radial dimension of said syringe head is not greater than the maximum radial dimension of said syringe body.

10. The syringe of claim 9, further comprising a closure for said syringe head and the maximum radial dimension of said syringe head including said closure is not greater than the maximum radial dimension of said syringe body.
